(19) United States
(12) Patent Application Publication
(10) Pub. No.: US 2023/0059527 A1
(43) Pub. Date: Feb. 23, 2023

(54) PACKAGING OF CULTURED TISSUE

(71) Applicant: Mosa Meat B.V., Maastricht (NL)

(72) Inventor: Peter VERSTRATE, Maastricht (NL)

(21) Appl. No.: 17/759,337

(22) PCT Filed: Feb. 3, 2021
(86) PCT No.: PCT/NL2021/050067

§ 371 Date: Jul. 22, 2022

(30) Foreign Application Priority Data
Feb. 3, 2020 (NL) 2024821

(51) Int. Cl. B65B 25/06; B65B 55/02; A23L 13/00; C12N 5/071

(52) U.S. Cl. CPC B65B 25/067; B65B 55/025; A23L 13/00; C12N 5/0697; B65B 2210/08; A23V 2002/00

(57) ABSTRACT

The invention is in the field of cultured meat. In particular the invention is related to a method for aseptic packaging of cultured tissue and a system suitable for this method. The method comprises the steps of producing the cultured tissue, harvesting the cultured tissue, transferring the cultured tissue to a sterile package and sealing the sterile package, wherein all steps are executed under aseptic conditions.

The invention is in the field of cultured meat. In particular the invention is related to a method for packaging cultured meat and a system suitable for this method.

In food industry it is important that products are packaged in a way that reflects their sensitivity to spoilage. Meat products, and especially fresh meat, are often very good substrates for bacterial growth and are consequently packed as aseptically as possible to minimize contamination. The term aseptic is used to indicate that a processing step is contamination-free, thus indicating that no microorganisms are added to the product. However, organisms that are already on the packaged meat prior to packaging remain intact so packaged meat that has not been post-treated with a sterilization method is never sterile. Sterilization is an aseptic technique to fully eliminate, remove or kill all microorganisms. Sterilization can be done through several means including heat, irradiation, chemical treatment, pressure and combinations thereof.

Aseptic packaging is the filling of a sterilized container with a sterilized product under aseptic conditions. It allows for sterilization of the product outside of the container, in contrast to methods wherein the filled package (viz. including the product) are simultaneously sterilized. Often the sterilization for aseptic packaging is performed through ultrahigh temperatures, meaning that the product is heated to a certain temperature for a specific time before it is allowed to cool down and to be packaged. In some cases, the product may remain at elevated temperatures to simplify filling of the container, due to for example the viscosity. The final step is to seal the package, often achieved using heat. The package is preferably sealed hermetically, in other words, airtight, to maintain sterility during handling and distribution of the product.

Aseptic packaging is widely used in the food and beverage industry to prolong shelf life. The aseptically packaged products can obtain a non-refrigerated shelf life up to several years. If the product is sterilized through ultra-high temperatures in a relatively short time, a high retention of nutritional value may even be achieved. However, the requirement of the sterilization step is also a drawback, as it is energetically costly and time consuming. Also, heat sterilization changes, in some cases adversely, the organoleptic properties of the product.

In particular meat is irreversibly changed by the application of heat: it is impossible to heat a meat product while retaining the fresh properties, and the same applies to the other techniques available for sterilization.

The materials used as aseptic or sterile packages should meet several requirements. The material should be compatible with the products to be packaged. The material should have physical integrity to properly contain the product and maintain sterile over a period of time. Furthermore, it is required that the material withstands sterilization methods. Finally, often the material should protect the product from oxygen and must retain the aroma of the product. This is especially relevant for vacuum packaging and modified atmosphere packaging (MAP). However, fresh meat products may be packaged at elevated oxygen levels to protect the color (i.e. to retain the oxygenation of myoglobin). Here the main requirement of the package is the need to protect against micro-organisms. Myoglobin may oxygenate into a deep oxymyoglobin at elevated oxygen concentrations, whereas myoglobin would oxidize into the brown metmyoglobin in ambient concentrations of oxygen. Oxidation is the chemical reaction reducing the Fe²⁺ molecule on myoglobin; the red version is oxygenized, meaning it carries (transports) the oxygen molecule but does not react with it. In the oxygenated state the myoglobin is red, in the oxidized state it is brown. Often packages are composed of several layers of different materials to meet the requirements. Examples of such materials may include polyethylene, aluminum, paper and combinations thereof.

Aseptic packaging in the meat industry presents several obstacles as the meat is by definition contaminated. The meat is highly vulnerable to spoilage as it provides an excellent substrate for growth of micro-organisms. Thus, rendering a limited shelf life and high product waste.

A sterilization method for meat can be heat treatment. Heat treatment may be conducted by placing the product in a hermetically sealed container, which is submerged in hot water, steam or a combination thereof. A temperature above 100° C. may be achieved under pressure. An alternative is to use temperatures up to 100° C. However, several microorganisms are resistant to the lower temperatures and therefore the meat must be stored under reduced temperatures. Another drawback is found in the need to cool the meat as quickly as possible to avoid overcooking of the product. The process to quickly cool the meat presents industrial challenges. Moreover, this process irreversibly changes the product and is not suitable for fresh meat.

Fresh meat typically has a very short shelf life of only 5-9 days. As it is a destination product, retailers make a lot of effort to have 100% service levels. Consequently, the waste is high due to a lot of over-code products. The waste at retailer and consumer level can be up to 30%.

Other options for packaging fresh products and physically protecting the product are, the previously mentioned, vacuum packaging and MAP. Vacuum packaging is a method that removes air from the package before sealing. An advantage of this method is that the method inhibits bacterial growth. However, as it does not eliminate microorganisms the shelf life remains limited even under reduced temperatures. A vacuum package will only be sterile if it is post-treated with a sterilization technique. MAP is considered more effective for meat as it may comprise a combination of gasses, usually N₂, CO₂, O₂. The CO₂ may dissolve on the surface of the meat resulting in a slower rate of spoilage as the growth of micro-organisms is retarded.

Prior to selling meat or using meat for consumption, a process to tenderize meat is performed. This process is called ageing and it works by breaking down connective tissue by natural enzymes such as cathepsin and calpain. Secondly glycogen, the natural sugar present in muscles, is converted into lactic acid thereby creating a lower pH of the meat. Ageing can be categorized in wet- and dry-ageing.

For higher end meats found at the butcher a dry-ageing process is preferred. After the slaughter of the animal, the carcass is hung in a refrigerated environment or climate-controlled environment and subjected to dry-ageing. The process takes place near freezing temperatures and is responsible for the concentration and saturation of the natural flavors and the tenderization of the texture. Dry-ageing works by evaporation of moisture from the muscle tissue to increase the concentration of the flavors. Furthermore, the natural enzymes present in the meat break down connective tissue, thus increasing the tenderness. The process takes up a significant amount of time, even up to three months for beef. Moreover, due to the evaporation of moisture there is a significant weight loss making the process less profitable.

Wet-ageing is a process in which meat is vacuum-sealed to retain the moisture content, thus maintaining a higher weight. The meat is usually kept in a climate-controlled or refrigerated container for up to ten days. Retailers, wholesalers and producers have a preference for this process as it is more profitable.

The final drying of the aged meat provides a more stable product as no micro-organisms grow on the dry products. However, the ageing process does not render the meat sterile.

As there are quite some drawbacks in the conventional meat industry, an alternative can be found in cultured meat. Cultured meat is produced from one or more mammalian cells, e.g. myosatellite cells, that are encouraged to grow and specialize into muscle cell tissue. The process of producing cultured meat includes sourcing, characterization, selection, proliferation and differentiation of the cells, and takes place ex vivo. The growth process is usually performed in a medium. This medium provides suitable chemical, topographical and mechanical properties for the myosatellite cells to grow and specialize into muscle tissue. The medium may be placed in a bioreactor. The myosatellite cells can potentially be obtained without the need to slaughter the animal. The muscle tissue may be harvested and may be used for human consumption. Up to the point of harvesting, the process takes place under controlled, sterile conditions.

Ageing of the cultured meat may be achieved via a process similar to the ageing of traditional meat. For example, it may be performed by a substitute such as a certain pH setting or enzyme treatment. Whereas ageing of traditional meat at is typically performed in a refrigerated environment, because higher temperatures would pose a risk of the development of spoilage bacteria, this is different for cultured meat. Because cultured meat is produced under sterile conditions, it may be possible to perform ageing of cultured meat at slightly elevated temperatures, such as ambient temperatures up to 40° C., provided that the sterile conditions are kept during the ageing process. Performing ageing at elevated temperatures could speed up the ageing process considerably. A challenge remains to find a method to package the material in such a manner to reduce product waste and to prolong storage. The storage default of a fresh product is packaging with a low level of contamination.

CN109567037 discloses a method to process crocodile meat. The method comprises a plurality of steps including sterilization steps using a low temperature at high pressure and UV irradiation. Where all steps are time consuming, UV irradiation moreover changes the vitamin content of the food. Also, UV alone has limited penetration and is insufficient for sterilization of meat.

JPH08308478 discloses a machine for sterilizing and aseptic packaging of meat products. The sterilization is performed by high temperature inside of a sealed chamber during sealing. The temperature reaches an average of 100° C. to 160° C. for 15 seconds or less.

A common drawback is the requirement of the sterilization step. The step is energetically costly, time consuming, possibly alters nutrient content and changes the overall properties of the product dramatically in the case of meat.

It is an object of the present invention to provide a method which at least in part overcomes the above-mentioned drawbacks.

The present inventors have surprisingly found that aseptic packaging and cultured tissue can be combined, eliminating the need for an additional sterilization step of the product before packaging.

Thus, in a first aspect, the present invention is directed to a method for aseptic packaging of cultured tissue. The method comprises the step of producing cultured tissue in a sterilized bioreactor. Further the method comprises the step of harvesting the cultured tissue from the sterilized bioreactor, the step of transferring the cultured tissue to a sterile package and the step of sealing the sterile package. Wherein the steps are executed under aseptic conditions. Only in aseptic conditions it is possible to produce cultured tissue without the addition of antibiotics. Contamination will be fatal to the cultured tissue, thus meaning no product can be obtained. The environment may be prone to contamination, therefore adequate contamination indicators are preferably present. The resources used for producing cultured tissue have preferably been sterilized before use. This is preferably without application of chemicals, heat or other effects, for instance using microfiltration or ultrafiltration. The produced cultured tissue may therefore be sterile. Preferably the cultured tissue is fresh cultured tissue, wherein fresh relates to the cultured tissue not being post-processed by means of heat suitable for a sterilization environment. Because the steps following the production of fresh cultured tissue, including the steps of packaging and sealing the cultured tissue are performed under aseptic conditions, packaged cultured tissue (e.g. meat) with a long shelf can be produced, without the need for additional sterilization treatment or preservative additives.

The method may further comprise one or more processing steps which are also carried out under aseptic conditions. Examples of such process steps include dewatering and/or spicing of the cultured tissue, and forming a shaped meat product (such as a hamburger patty) from a paste.

In a preferred embodiment, the sterile package comprises a sterile chamber. The sterile package is preferably hermetically sealed. Sealing may be done by conventional methods including heat sealing using a thermoplastic, mechanical closing of a lid, clamping force and/or cold welding. The package preferably includes a manner of analysis for quality assurance. This may be in the form of an indicator for the integrity of the packaging. Examples include a patch that discolors in the presence of oxygen to indicate a leaking package, this may be combined with a patch that changes color if there are bacterial anaerobic metabolites present.

Under certain circumstances a MAP comprising oxygen may be preferred. For these cases it is not preferred to have a patch that discolors in the presence of oxygen. An alternative may be in the form of in-line quality checks. By passing the packages through a vacuum, thereby measuring the difference in pressure can be used to determine if the integrity of the package is breached. In a preferred embodiment the method comprises an integrity test of the sterile package, this is preferably in the form of any of the above-mentioned manners. The integrity test may show breaching of the package rendering it insufficient for adequate aseptic packaging.

Aseptic conditions are preferably maintained via conventional means such as sterilization of equipment and environmental control. The sterilization of equipment is of importance to eliminate, kill and remove all microorganisms before the equipment is used in the aseptic process. The environmental control is to ensure no contact between the aseptic part and the outer environment takes place. For maximum food safety it is preferred to have a grade A Isolator with self-sterilization, e.g. following EC Good Manufacturing Practice (GMP), Standard I.

The bioreactor preferably comprises a medium suitable for the formation of cultured tissue. It may be suitable if the medium provides sufficient chemical, topographical and structural features. In a preferred embodiment, the medium is in the form of a hydrogel. The medium preferably comprises a polysaccharide, such as an alginate.

In embodiments, all steps of the method as described herein are performed in the same sterile enclosure.

It is also possible that each step or at least some steps are performed in separate sterile enclosures. In that case, transferring cultured tissue between the separate enclosures is performed under aseptic conditions.

The one or more enclosures preferably comprise a grade A isolator capable of self-sterilization. For instance, if all steps are performed in the same sterile enclosure such as a grade A isolator capable of self-sterilization, this would simplify filling as a connecting piece between the package and the filling machine does not have to be hermetically connected. The post-processing and packaging can therefore be performed under strictly aseptic conditions.

The method for aseptic packaging of cultured tissue may further comprise the step of transferring the cultured tissue to a sterilized connecting piece and the step of transferring the cultured tissue from the sterilized connecting piece to the sterile package. The sterilized connecting piece preferably connects the bioreactor of cultured tissue with the sterile package. The steps are executed under aseptic conditions. The connection is preferably hermetic. In a preferred embodiment the connection piece connects the bioreactor to a sterile chamber of the sterile package.

The method of packaging may for instance comprise using a three-valve system with a steam connection and a condensate drain. The middle valve may be a split butterfly valve, or similar. After connecting, the split valve may be opened and the area between the external valves can be sterilized by steam sterilization. After draining the condensate and cooling of the connection the two other valves can be safely opened, without risk of infection. These would discharge into vessels that have been sterilized. Transfer of the tissue may for example be carried out in suspension.

In another preferred embodiment, the steps of the method are executed in the bioreactor under aseptic conditions. This may be in the form that the sterile package is introduced into the bioreactor. Preferably the medium is introduced in the sterile package. The medium may be removed before sealing of the sterile package. Removal may be achieved by enzymatic degradation or washing with a sterile post processing solution. The post processing solution may be flavored.

By executing the steps in the bioreactor, the risk of contamination may be reduced. Moreover, by providing a single process it may be energetically more favorable and less time consuming.

In a preferred embodiment the individual elements involved in the method are easily sterilized. It may be required to sterilize the sterile package, sterilized connecting piece and sterilized bioreactor. Preferably conventional sterilization methods such as heat, irradiation, chemical treatment and combinations thereof are sufficient to sterilize the individual elements. Furthermore, the ingredients of the growth medium are preferably sterilized. Sterilization may be achieved through e.g. sterilization filtration.

The cultured tissue is preferably cultured meat. The cultured tissue may be grown in such a manner to provide muscle cell tissue, that is preferably suitable to be aseptically packaged. More preferably the cultured meat is suitable for consumption. As used herein, “suitable for consumption” refers to the suitability for consumption by humans and/or animals, preferably by humans. It may be suitable for consumption if the myosatellite cell originates from cows, sheep, pigs, poultry, fish or the like, and combinations thereof. The combination of cultured meat and packaging under aseptic conditions may extend shelf life and reduces or may even eliminate over-code products. This extends to retailers but also to consumers.

In a preferred embodiment the process pressure within the bioreactor is between 0 to 11 bar absolute pressure. The process pressure relates to the pressure present during the process, this process may comprise all the steps of the method or may comprise one or more steps. The pressure influences the aseptic conditions in combination with the temperature. Depending on the temperature the pressure is at the lower or higher end of the range. Pressure alone cannot sterilize a product. The pressure may be sufficient for correct growing of the cultured tissue. Furthermore, the pressure may be sufficient to maintain aseptic conditions.

Preferably the process temperature in the bioreactor is between 0 to 50° C. The process temperature relates to the temperature present during the process, this process may comprise all the steps of the method or may comprise one or more steps. The process temperature may be dependent on the pressure. The temperature range is well below temperatures for sterilizing conditions. It may be preferred to have a process temperature of 20 to 40° C. in case the step comprises production of the cultured tissue. The temperature is of importance for the sufficient growing conditions for the cultured tissue. Furthermore, the temperature may be sufficient to maintain aseptic conditions.

In another preferred embodiment the cultured tissue is transferred to the sterile package at a temperature between 0 to 50° C. The temperature may be chosen dependent on the material. A higher temperature may influence the tissue. At higher temperatures the tissue may be partly cooked, and the structural features may change. Irreversibly altering the integrity of the tissue is then inevitable. Moreover, a higher temperature may damage the physical integrity of the packaging material. The physical integrity of the packaging material may also be influenced by a lower temperature. The temperature furthermore is significantly lower than needed for common sterilization conditions.

Preferably the sterile package is impermeable to oxygen. This sterile package may be in the form of a blister package with a recyclable barrier film, or it may be a reusable glass or metal container.

The cultured tissue is preferably transferred to the sterile package at a pressure between 0 to 5 bar absolute pressure. The pressure may be sufficient for time effective transfer. Moreover, the pressure may be sufficient to correctly fill the sterile package. It may be preferred to use protective atmosphere during the transferring of the cultured tissue to the sterile package.

Preferably the sterile package is suitable for storage between −20 to 50° C. A higher temperature may alter the shelf life as well as the taste. The package may be suitable for storage under refrigerated conditions as well as ambient conditions, thereby still needing to fulfill all requirements for aseptic packaging. At lower temperatures, the structural integrity of the material may be compromised.

An aseptic packaging system may be designed for the aseptic packaging of cultured tissue. This system comprises a sterilized bioreactor for producing cultured tissue, a sterile package and a means for sealing the sterile package. The system preferably can operate under aseptic conditions. In a preferred embodiment the system comprises a grade A isolator capable of self-sterilization. The aseptic system packaging system is preferably suitable for aseptic packaging of fresh cultured tissue.

In accordance with the invention it is possible to produce fresh mincemeat or fresh (viz. uncooked) hamburgers with a shelf life of up to 60 days.

Another advantage of aseptic packaging according to the invention is that less preservatives such as nitrites and nitrates have to be added to the meat. Preservatives such as nitrites and nitrates are typically added to certain meat products, such as ham, inter alia to prevent growth of bacteria such as Clostridium botulinum, which causes botulism. Because of the aseptic conditions during production and packaging of the cultured tissue, there will be much less chance of infection with these bacteria. Therefore, less or no preservatives have to be added. For example, the amount of sodium nitrite needed to prevent the occurrence of botulism may be less than 50 mg/kg meat, preferably less than 10 mg/kg. Still more preferably, the occurrence of botulism can be prevented when the meat is free or substantially free of sodium nitrite. Sodium nitrite also acts as a colorant, e.g. giving ham its pink color. If less or no sodium nitrite is used, other, less toxic colorants may be used instead.

Because of the aseptic conditions during production and packaging of the cultured tissue, ageing of the cultured tissue can be performed at elevated temperatures, because growth of unwanted micro-organisms at elevated temperatures is not a concern. This means that ageing does not have to be performed near freezing temperatures and/or in a refrigerated container. For instance, ageing can be performed at temperatures of 5° C. or higher, such as 10-50° C., for instance at room temperature. The cultured tissue can be aged before packaging and/or after packaging.

It is also possible to deliberately add certain micro-organisms to the cultured tissue. For instance, if fermentation or ageing of the cultured tissue under the influence of specific micro-organisms is desired, such micro-organisms can be added. The cultured tissue can for instance be fermented using micro-organisms before packaging. Because the aseptic conditions result in the absence of unwanted and harmful micro-organisms, the desired microbial process (such as fermentation) becomes predictable and controlled. In order to prevent these deliberately added micro-organisms to contaminate the bioreactor, a step of deliberately adding micro-organisms should not be performed in the same enclosure as the bioreactor. Despite the addition of micro-organisms, the steps of the method can still be considered to have been executed under aseptic conditions, because the only micro-organisms that will be present are deliberately added, and the introduction of unwanted and harmful micro-organisms is still prevented. An example of a meat product in which micro-organisms are deliberately added is salami.

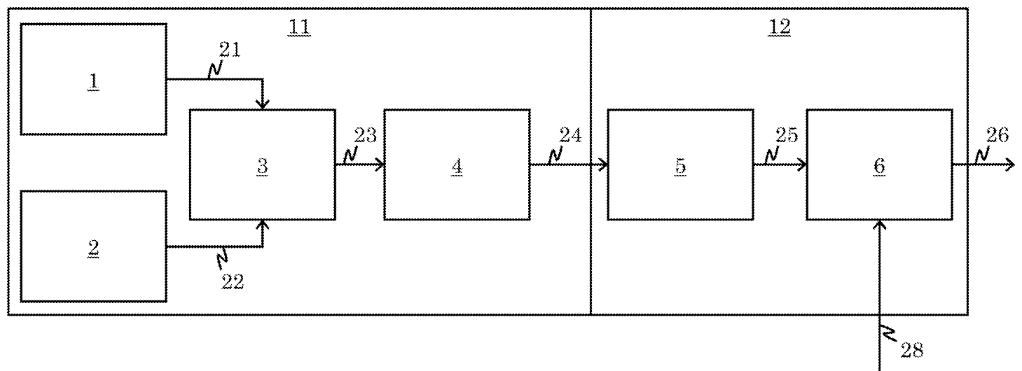

An example of a method in which hamburgers made from cultured tissue suspensions are aseptically packaged is schematically shown in FIG. 1 and described below. However, the invention is not limited to hamburgers or other meat products that are made from tissue suspensions. It may also possible to produce larger (e.g. portion-sized) pieces of tissue in the bioreactor that can be packaged individually. Similar process steps may also apply to other products than hamburgers.

With reference to FIG. 1, muscle tissue is produced in muscle tissue bioreactor array (1), and fat tissue is produced in fat tissue bioreactor array (2). Muscle tissue suspension (21) and fat tissue suspension (22) are led to buffer tank (3), which provides a continuous feed for the subsequent steps. Mixed tissue suspension (23) is led to dewatering and spicing system (4), where the suspension is dewatered and where sterile spices and/or additives (27) can be added. After that, hamburger paste (24) is led to hamburger forming station (5), where it is formed into hamburger (25). Hamburger (25) is then packaged in packaging station (6) using sterile packaging material (28) and the package is sealed. Packaged hamburger (26) can then be removed from aseptic conditions and further processed on a standard packaging line.

The cultured tissue that is produced in bioreactor arrays (1) and (2) is free of micro-organisms, and the further processing and or packaging steps are carried out under aseptic conditions. Therefore, aseptically packaged cultured tissue can be obtained without the need for sterilizing the tissue. Items that are introduced from outside the aseptic environment, such as spices and/or additives (27) and packaging material (28) have to be sterilized when they are brought into the aseptic environment in order to avoid contamination.

In the example of FIG. 1, muscle tissue and fat tissue are produced as aseptic suspensions, meaning that fluid streams are obtained from the bioreactor arrays. The steps involving a fluid stream are performed under aseptic conditions in fluid zone (11). The units in this zone, such as process vessels (i.e., tanks) and fluid transfer lines can be cleaned and sterilized in a cleaning cycle. To this end, the equipment in fluid zone (11) may be equipped with clean-in-place (CIP) and sterilization-in-place (SIP) systems. The vessels and fluid transfer lines may for instance comprise several inlets and outlets through which cleaning liquids can introduced and removed from the system. After cleaning with a cleaning liquid, the process vessels and lines can be sterilized with e.g. steam. Therefore, the equipment in fluid zone (11) is preferably built from material that can withstand the conditions of the sterilization process, for instance high temperatures and pressures in case of sterilization with steam. The fluids can be transported using low shear stress pumps, that are preferably easily cleanable. For example, peristatic pumps or membrane pumps can be used.

After dewatering of the mixed tissue suspension, further steps take place in an aseptic environment outside the fluid zone, for instance in grade A isolator (12). The boundary between fluid zone (11) and grade A isolator (12) can for instance be formed by a three-way valve. Such a three-way valve can also be used as inlet and/or outlet for a CIP process.

Grade A isolators are hermetically sealed isolators that circulate air through HEPA filters to keep the environment sterile. Any operator contact with the systems in such an isolator is done with gloves. In addition, Grade A isolators are typically kept under overpressure with respect to the surrounding atmosphere, to prevent gas and microorganisms from the surrounding atmosphere from entering the isolator. Before production runs the isolator can be flooded with ca. 150 ppm of vaporized hydrogen peroxide. This kills all microorganisms and sterilizes the isolator. In this way it provides a contamination free workspace for aseptic processing. Afterwards, the vaporized peroxide is removed, typically using a catalytic filter. Production is only started if the peroxide levels fall below levels which are safe for the product, thereby preventing contamination and degradation of the food product by hydrogen peroxide.

Because the output of tissue bioreactors is typically periodic, arrays of bioreactors, such as muscle tissue bioreactor array (1) and fat tissue bioreactor array (2) can advantageously be used to provide a continuous feed for the subsequent steps.

Buffer tank (3) can be a simple stirred tank that acts as a buffer for the output of the bioreactors. Its purpose is to provide a continuous feed for the packaging line. In addition, muscle tissue suspension (21) and fat tissue suspension (22) can be mixed in the buffer tank.

Dewatering and spicing system (4) can be implemented as a two-stage process in which the mixed tissue suspension is continually dewatered using, for example, a belt filter press which feeds into a mixing auger. Sterile spices and/or additives (27) can be added in the mixing auger, and the tissue spice mixture is treated to get the desired texture.

The output of dewatering and spicing system (4) is hamburger paste (24). This paste can be transported by the mixing auger to the hamburger forming station (5), which for instance comprises a forming wheel in which the hamburgers are formed. The formed hamburger (25) can then be fed onto a belt, from where it can be placed into packaging station (6) by a pick-and-place machine.

Packaging station (6) can for instance be a blister machine which seals hamburgers into thermoformed packages under a protective atmosphere. To this end, sterile packaging material (28), for instance a stiff thermoformable film and a flexible sealing film, can be brought into the isolator through a sterilizing inlet. Sterilization of the packaging material (for example UV sterilization) is used to ensure that no contamination can enter the isolator. The stiff film is then heated, and vacuum formed into the desired container shape. Hamburger (25) can be placed inside the formed product cavities. The container is then sealed in a sealing step and the sealed container is cut from the packaging material and brought out of the isolator in a controlled manner without contaminating the isolator, for instance using an aseptic airlock. From this point, since the products are aseptically packaged and the package is hermetically sealed, the packaged product can be further processed in a standard packaging line without measures to maintain aseptic conditions.

All devices and parts in the isolator should be designed in such a way that they can be easily disassembled and cleaned in between packaging runs. The disassembled parts should then be placed in positions in which good contact with the sterilization vapors, e.g. vaporized hydrogen peroxide (VHP), can be ensured. An operator can then reinstall all parts before a packaging run without opening the isolator and breaking sterility.

There are several ways in which the quality of the packaged products can be checked. Periodic samples can be taken to confirm the absence of microbes. Packaged products can be stored for several days and checked for signs of contamination. Chemical indicators that can detect undesired microbiological growth can be included in the packaging. Yearly runs with an indicator product can be performed in order to confirm that the process can be run without introducing contamination.

For the purpose of clarity and a concise description, features are described herein as part of the same or separate embodiments, however, it will be appreciated that the scope of the invention may include embodiments having combination of all or some of the features described.

1-22. (canceled)

23. A method for aseptic packaging of cultured tissue comprising the steps of:
producing cultured tissue in a sterilized bioreactor;
harvesting cultured tissue from said sterilized bioreactor;
transferring cultured tissue to a sterile package;
sealing said sterile package;
wherein the steps are executed under aseptic conditions, and
wherein the cultured tissue is cultured meat for consumption.

24. The method according to claim 23, further comprising one or more processing steps which are also carried out under aseptic conditions.

25. The method according to claim 23, wherein all steps are performed in the same sterile enclosure.

26. The method according to claim 23, wherein at least some of the steps are performed in separate sterile enclosures and wherein transferring cultured tissue between the separate enclosures is performed under aseptic conditions.

27. The method according to claim 23 further comprising the steps of:
transferring cultured tissue to a sterilized connecting piece;
transferring cultured tissue from said sterilized connecting piece to said sterile package;
wherein the steps are executed under aseptic conditions and wherein said sterilized connecting piece connects said sterile package with said sterilized bioreactor of cultured tissue.

28. The method according to claim 23 wherein the steps are executed in said sterilized bioreactor.

29. The method according to claim 23 wherein the one or more enclosures comprise a grade A isolator capable of self-sterilizing.

30. The method according to claim 25 wherein said enclosure is a grade A isolator capable of self-sterilizing.

31. The method according to claim 26 wherein said separate sterile enclosures are grade A isolators capable of self-sterilizing.

32. The method according to claim 23 wherein said method further comprises an integrity test of the sterile package.

33. The method according to claim 23 wherein the process pressure in said sterilized bioreactor is between 0 to 11 bar absolute pressure.

34. The method according to claim 23 wherein the process temperature in said sterilized bioreactor is between 0 to 50° C.

35. The method according to claim 23 wherein cultured tissue is transferred to said sterile package at a temperature between 0 to 50° C.

36. The method according to claim 23 wherein cultured tissue is transferred to said sterile package at a pressure between 0 to 4 bar absolute pressure.

37. The method according to claim 23 wherein the sealing of said sterile package is hermetic.

38. The method according to claim 23 wherein the sterile package comprises a sterile chamber.

39. The method according to claim 23 wherein said sterile package is suitable for storage between −20 to 50° C.

40. The method according to claim 23 wherein said sterile package, said sterilized connecting piece and said sterilized bioreactor of cultured tissue are easily sterilized by conventional sterilization methods.

41. The method according to claim 23 wherein the bioreactor comprises a medium suitable for the formation of cultured tissue.

42. The method according to claim 41 wherein the medium is a hydrogel.

43. The method according to claim 23 wherein cultured tissue is fresh cultured tissue.

44. The aseptic packaged cultured meat obtainable by the method according to claim 23.

45. The aseptic packaged cultured meat according to claim 44 having a shelf-life of up to 60 days.

46. An aseptic packaging system for aseptic packaging of cultured tissue according to claim 23, wherein the cultured tissue is cultured meat for consumption, said aseptic packaging system comprising a sterilized bioreactor for producing cultured tissue, a sterile package and a means for sealing said sterile package, wherein the system operates under aseptic conditions.
